(19) United States
(12) Patent Application Publication
(10) Pub. No.: US 2026/0335187 A1
(43) Pub. Date: Oct. 29, 2026

(54) CLOSURE FOR PHARMACEUTICAL PREPARATIONS AND METHOD AND ROTARY DEPOSITION APPARATUS FOR MANUFACTURING THEREFOR

(71) Applicant: Applied Materials, Inc., Santa Clara, CA (US)

(72) Inventors: Jose Manuel DIEGUEZ-CAMPO, Hanau (DE); Vibhas SINGH, San Jose, CA (US); Neil MORRISON, Darmstadt (DE)

(21) Appl. No.: 19/106,354

(22) PCT Filed: Sep. 7, 2022
(86) PCT No.: PCT/EP2022/074900

§ 371 Date: Feb. 25, 2025

(51) Int. Cl. A61J 1/14; B65D 39/04; B65D 41/28; B65D 81/24

(52) U.S. Cl. CPC A61J 1/1406; A61J 1/1412; B65D 39/04; B65D 41/28; B65D 81/24; B65D 2251/20

(57) ABSTRACT

A closure for pharmaceutical preparations, a method for manufacturing closures for pharmaceutical preparations, and a rotary deposition apparatus for manufacturing closures for pharmaceutical preparations are provided. The closure for pharmaceutical preparations includes a polymeric closure body and at least one barrier layer over the polymeric closure body. Further, the at least one barrier layer comprises an adhesion promotion layer material within at least a portion of a thickness of the at least one barrier layer proximal to a surface of the polymeric closure body and the adhesion promotion layer material includes SiOxCy:H. Furthermore, the at least one barrier layer comprises a barrier layer material within at least a portion of the thickness of the at least one barrier layer distal from the surface of the polymeric closure body and the barrier layer material is selected from the group consisting of SixNy, SixCy, SixOyNz, SixOyCz, SiO₂, AlxOy, AlOxNy, TiO₂, and combinations thereof.

### TECHNICAL FIELD

Embodiments of the present disclosure relate to a closure for pharmaceutical preparations, a method for manufacturing closures for pharmaceutical preparations, and a rotary deposition apparatus for manufacturing closures for pharmaceutical preparations.

### BACKGROUND ART

In recent times, development of new pharmaceutical preparations including monoclonal antibodies, cell and gene therapies, vaccines, and recombinant proteins and peptides and thereby corresponding marketing authorizations have increased rapidly. In this regard, one of the most remarkable examples in the last few years is the development and consequent introduction of COVID-19 vaccines in the market.

Protecting such pharmaceutical preparations from undesired interactions with a surrounding environment and preserving the efficacy thereof during shelf life is one of the most pressing challenges for the pharmaceutical industry.

Pharmaceutical preparations, particularly biological pharmaceutical preparations, are mainly offered as liquid or lyophilized formulations in container systems, e.g., glass containers closed with polymeric closures, which are also known as parental closures or closures for pharmaceutical preparations. Synthetic rubber closures are the first choice for parental closures because of their higher purity and remarkable material properties, particularly their elasticity. The elasticity leads to a tighter contact with the glass container and thus ensures integrity and sterility of a contained pharmaceutical preparation. Furthermore, rubber has a sufficient resealing capability that allows repeated piercing by a needle. Accordingly, closures for pharmaceutical preparations can be found in the form of stoppers, plungers, and needle shields.

In order to achieve necessary material properties of the closures for pharmaceutical preparations, additives like plasticizers, fillers, antioxidants, and pigments must be added during manufacturing thereof. These substances have no covalent binding to polymer chains of the closures for pharmaceutical preparations and consequently may migrate from the closures into the pharmaceutical preparations themselves during their shelf life. A migration of substances, also referred as extractables or leachables, should be minimized as a migration of this sort can negatively affect the pharmaceutical preparation's safety, stability, and efficacy.

With the foregoing in view, there is a need for closures for pharmaceutical preparations with improved barrier properties against migration of substances, e.g., extractables or leachables, and also with water vapor and oxygen barrier properties, which also help to preserve the desired properties of the pharmaceutical preparations for a longer time. Furthermore, there is a need for methods for manufacturing such closures for pharmaceutical preparations which are relatively easy and cost-effective to perform and which provide such closures for pharmaceutical preparations with the above-mentioned properties. Furthermore, there is a need for an apparatus for manufacturing such closures for pharmaceutical preparations, which allows these closures for pharmaceutical preparations to be manufactured on an industrial scale and at a high-productivity level.

### SUMMARY

In light of the above, a closure for pharmaceutical preparations, a method for manufacturing closures for pharmaceutical preparations, and a rotary deposition apparatus for manufacturing closures for pharmaceutical preparations are provided. Further aspects, advantages, and features of the present disclosure are apparent from the claims, the description, and the accompanying drawings.

According to an aspect of the present disclosure, a closure for pharmaceutical preparations is provided. The closure for pharmaceutical preparations includes a polymeric closure body and at least one barrier layer over the polymeric closure body. Furthermore, the at least one barrier layer comprises an adhesion promotion layer material within at least a portion of a thickness of the at least one barrier layer proximal to a surface of the polymeric closure body, the adhesion promotion layer material including SiOxCy:H. Furthermore, the at least one barrier layer comprises a barrier layer material within at least a portion of the thickness of the at least one barrier layer distal from the surface of the polymeric closure body, the barrier layer material being selected from the group consisting of SixNy, SixCy, SixOyNz, SixOyCz, SiO₂, AlxOy, AlOxNy, TiO₂, and combinations thereof.

According to another aspect of the present disclosure, a method for manufacturing closures for pharmaceutical preparations is provided. The method includes: providing at least one polymeric closure body and forming at least one barrier layer over the at least one polymeric closure body. In addition, forming at least one barrier layer over the at least one polymeric closure body includes depositing an adhesion promotion layer material and depositing at least one barrier layer material in this order. Furthermore, the at least one barrier layer comprises the adhesion promotion layer material within at least a portion of a thickness of the at least one barrier layer proximal to a surface of the at least one polymeric closure body, the adhesion promotion layer material including SiOxCy:H. Furthermore, the at least one barrier layer comprises at least one barrier layer material within at least a portion of the thickness of the at least one barrier layer distal from the surface of the at least one polymeric closure body, the barrier layer material being selected from the group consisting of SixNy, SixCy, SixOyNz, SixOyCz, SiO₂, AlxOy, AlOxNy, TiO₂, and combinations thereof.

According to a further aspect of the present disclosure, a rotary deposition apparatus for manufacturing closures for pharmaceutical preparations is provided. The rotary deposition apparatus includes: a vacuum chamber, a rotary container located inside the vacuum chamber and configured to hold at least one polymeric closure body inside; and at least one deposition source connected to the vacuum chamber, located outside of the vacuum chamber, and configured for depositing at least one barrier layer on the polymeric closure body. Further, the rotary container includes a perforated wall configured to allow deposition of the at least one barrier layer on the polymeric closure body.

### BRIEF DESCRIPTION OF THE DRAWINGS

So that the manner in which the above recited features of the present disclosure can be understood in detail, a more particular description of the disclosure, briefly summarized above, may be had by reference to embodiments. The accompanying drawings relate to embodiments of the disclosure and are described in the following:

FIG. 1 shows an example of a closure for pharmaceutical preparations and a corresponding schematic cross-sectional view of a portion of the closure for pharmaceutical preparations according to embodiments described herein;

FIGS. 2 to 5 show schematic cross-sectional views of a portion of a closure for pharmaceutical preparations according to embodiments described herein;

FIG. 6 shows a flow chart of a method for manufacturing closures for pharmaceutical preparations according to embodiments described herein;

FIGS. 7 and 8 show schematic views of a rotary deposition apparatus for manufacturing closures for pharmaceutical preparations according to embodiments described herein; and

FIG. 9 shows a schematic view of a rotary container of a rotary deposition apparatus for manufacturing closures for pharmaceutical preparations according to embodiments described herein.

### DETAILED DESCRIPTION OF EMBODIMENTS

Reference will now be made in detail to the various embodiments of the disclosure, one or more examples of which are illustrated in the figures. Within the following description of the drawings, the same reference numbers refer to the same features. Generally, only the differences with respect to individual embodiments are described. Each example is provided for explaining of the disclosure and is not meant as a limitation of the disclosure. Furthermore, features illustrated or described as part of one embodiment can be used on or in conjunction with other embodiments to yield yet a further embodiment. It is intended that the description includes such modifications and variations.

The rapid increase in the development, and thereby marketing authorizations, for pharmaceutical preparations including monoclonal antibodies, cell and gene therapies, vaccines, and recombinant proteins and peptides require further improvements in the production of container systems for pharmaceutical preparations in order to cover the high demand of such container systems.

Protecting pharmaceutical preparations from undesired interactions with a surrounding environment and preserving their efficacy during shelf life constitute one of the most pressing challenges for the pharmaceutical industry.

Pharmaceutical preparations, particularly biological pharmaceutical preparations, are mainly offered as liquid or lyophilized formulations in container systems, e.g., glass containers that are closed with polymeric closures, which are also known as parental closures or closures for pharmaceutical preparations. Closures for pharmaceutical preparations, particularly synthetic rubber closures, are commonly chosen because of their higher purity and remarkable material properties, particularly their elasticity. The elasticity leads to a tighter contact with the glass container and thus ensures integrity and sterility of a contained pharmaceutical preparation.

Since such container systems are rendered sterile before being placed on the market, preservation of seal integrity is itself vital for the preservation of sterility of the pharmaceutical preparation until the time the pharmaceutical preparation is administered to a patient. Accordingly, closures for pharmaceutical preparations can be found as stoppers, plungers, and needle shields in container systems for pharmaceutical preparations.

In order to achieve necessary material properties of the closures for pharmaceutical preparations, additives like plasticizers, fillers, antioxidants, and pigments must be added during manufacturing of the closures for pharmaceutical preparations. These substances have no covalent binding to polymer chains of the closures for pharmaceutical preparations and consequently may migrate from the closures for pharmaceutical preparations into the pharmaceutical preparations during shelf life of the pharmaceutical preparations. The migration of such substances, also referred as extractables or leachables, should be minimized because the migration may affect the pharmaceutical preparation's safety, stability, and efficacy.

Since the migration from the closures for pharmaceutical preparations may result in such disadvantages, closures for pharmaceutical preparations with improved performance, particularly with barrier properties against extractables or leachables, are desired. Further, the closures for pharmaceutical preparations are also expected to exhibit water vapor and oxygen barrier properties, which also help to preserve the desired properties of the pharmaceutical preparations for a longer time.

With the foregoing in view, in addition to improving the production of container systems for pharmaceutical preparations in order to satisfy the high demand of such container systems in the market, there is also a need for further improvements in the barrier properties, e.g., barrier properties against extractables or leachables, water vapor, and oxygen, of the closures for pharmaceutical preparations.

Furthermore, there is also a need for methods for manufacturing such closures for pharmaceutical preparations which are relatively easy and cost-effective to perform and provide such closures for pharmaceutical preparations with the above-mentioned properties. Furthermore, there is a need for an apparatus for manufacturing such closures for pharmaceutical preparations which allows such closures for pharmaceutical preparations to be manufactured on an industrial scale and at a high-productivity level.

The present disclosure fulfills the above-mentioned needs by providing a closure for pharmaceutical preparations, a method for manufacturing closures for pharmaceutical preparations, and a rotary deposition apparatus for manufacturing closures for pharmaceutical preparations. In particular, since the closure for pharmaceutical preparations of the present disclosure includes at least one barrier layer comprising an adhesion promotion layer material comprising SiOxCy:H and a barrier layer material selected from the group consisting of SixNy, SixCy, SixOyNz, SixOyCz, SiO₂, AlxOy, AlOxNy, TiO₂, and combinations thereof, the closure for pharmaceutical preparations of the present disclosure shows improved barrier properties, e.g., barrier properties against extractables or leachables, water vapor, and oxygen.

Furthermore, due to the location of the adhesion promotion layer material and the barrier layer material within the at least one barrier layer, the at least one barrier layer shows an appropriate balance between flexural rigidity and crack onset strain, i.e., fracture toughness, and thus the at least one barrier layer can undergo significant levels of mechanical strain and match mechanical properties of the polymeric closure body. In particular, the adhesion promotion layer material provides an intermediate region between the barrier layer material and the polymeric closure body that functions as a mechanical cushioning for mechanical strains.

Furthermore, due to the simplicity of the deposition of the at least one barrier layer on the polymeric closure body, the method for manufacturing closures for pharmaceutical preparations of the present disclosure allows a reduction of the production costs and time, and permits modulation of the barrier properties of the closure for pharmaceutical preparations by depositing further barrier layers with different barrier layer materials.

Moreover, the rotary deposition apparatus for manufacturing closures for pharmaceutical preparations of the present disclosure allows the closures for pharmaceutical preparations to be manufactured on an industrial scale, i.e., at a high-volume manufacturing level and with a high-productivity level. In addition, since the rotary deposition apparatus includes a rotary container causing a polymeric closure body or polymeric closure bodies to rotate inside the rotary container during manufacture of the closures for pharmaceutical preparations, deposition of the at least one barrier layer over the polymeric closure body is homogenous over the whole polymeric closure body. Further, deposition of the at least one barrier layer is carried out even on the portions of a surface of the polymeric closure body or polymeric closure bodies which are most difficult to access, due to the combination of the rotary container and the at least one deposition source.

Before various embodiments of the present disclosure are described in more detail, some aspects regarding some terms and expressions used herein are explained.

The term “pharmaceutical preparation” as used herein particularly refers to drugs presented in their finished dosage form, e.g., intended for human or veterinary use and, particularly, for treatment of illness or injury. The term “pharmaceutical preparation” as used herein particularly may also include materials used for preparation and/or formulation of a finished dosage form of a drug, e.g., intended for human or veterinary use and, particularly, for treatment of illness or injury.

Particularly, the term “drug” as used herein includes vaccines, antibodies, cells, gene, blood factors, proteins, recombinant proteins, peptides, and hormones, e.g., intended for human or veterinary use and, particularly, for treatment of illness or injury. Particularly, the term “drug” as used herein particularly may also refer to any natural or artificially-made chemical, e.g., intended for human or veterinary use and, particularly, for treatment of illness or injury.

In particular, the term “polymeric closure body” may be understood as a seal made of polymers that is used for closing any type of container, e.g., vials, bottles or syringes made of glass or plastic, used for pharmaceutical preparations. Particularly, the term “polymeric closure body” may refer to stoppers, plungers, and/or needle shields made of polymers that are used for closing any type of containers, e.g., vials, bottles or syringes made of glass or plastic, used for pharmaceutical preparations.

The term “elastomer” or “elastomeric” refers particularly to a natural or synthetic polymer having elastic properties, i.e., able to withstand large degrees of elastic deformation. Examples of polymers, particularly elastomers, used or included in a polymeric closure body or in polymeric closure bodies, particularly elastomeric closure bodies, are copolymers of styrene and butadiene, polyisoprene, and halogenated copolymers of isobutylene and isoprene such as bromobutyl rubber or chlorobutyl rubber.

The term “barrier layer” may particularly be understood as a layer made of a barrier layer material, particularly formed on a substrate, e.g., on a polymeric closure body, and more particularly on an elastomeric closure body. The term “barrier layer” may particularly be understood as a layer made of a barrier layer material, particularly formed on a substrate surface, e.g., on a surface of a polymeric closure body, and more particularly on a surface of an elastomeric closure body. In particular, the term “barrier layer” may also be understood as a layer made of a barrier layer material, said layer including barrier properties, e.g., against migration of extractables or leachables from a polymeric closure body, particularly an elastomeric closure body, to water vapor, and to oxygen.

In particular, the term “extractables” as used herein refers to organic and/or inorganic chemicals that are released from a polymeric closure body, particularly an elastomeric closure body, under laboratory conditions, i.e., under the influence of accelerated or exaggerated temperatures, solvents or surface exposure. Particularly, the term “leachables” as used herein refers to organic and/or inorganic chemicals that are released from a polymeric closure body, particularly an elastomeric closure body, during storage of pharmaceutical preparations under normal temperature and/or pressure conditions of storage of the pharmaceutical preparations.

Particularly, the term “gradient chemical composition” as used herein refers to a chemical composition that changes gradually, e.g., across a thickness of a layer, particularly in a vertical direction relative to a surface of a polymeric closure body.

When reference is made to the terms “on”, “over”, or “followed by”, e.g., at least one barrier layer over a polymeric closure body or a second barrier layer over a first barrier layer, it is understood that the terms “on”, “over”, or “followed by” are used to define an order of layers, wherein the starting point is a substrate, i.e., a polymeric closure body. This order is irrespective of whether or not the closure for pharmaceutical preparations, e.g., including the at least one barrier layer, is depicted upside down.

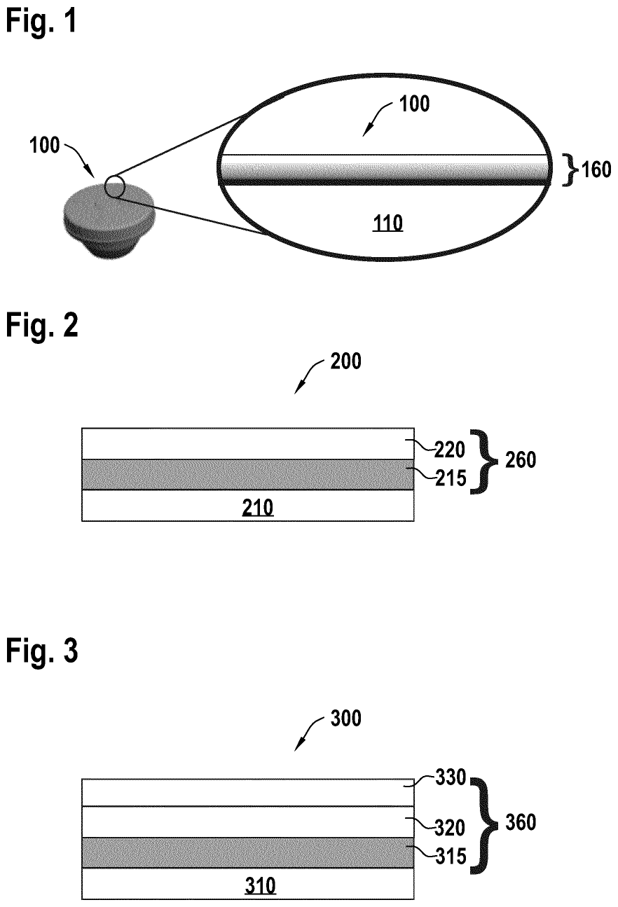

FIG. 1 shows an example of a closure for pharmaceutical preparations 100 and a corresponding schematic cross-sectional view of a portion of the closure for pharmaceutical preparations 100 according to embodiments described herein.

Although various embodiments of the present disclosure are described, for the sake of simplicity, with reference to the closure for pharmaceutical preparations 100, the various embodiments referring to the closure for pharmaceutical preparations 100 of the present disclosure may also apply or refer to the further closures for pharmaceutical preparations described in the present disclosure.

According to some embodiments, which can be combined with other embodiments described herein, the closure for pharmaceutical preparations 100 includes a polymeric closure body 110. Further, the closure for pharmaceutical preparations 100 includes at least one barrier layer 160 over the polymeric closure body 110. Furthermore, the at least one barrier layer 160 comprises an adhesion promotion layer material within at least a portion of a thickness of the at least one barrier layer 160 proximal to a surface of the polymeric closure body 110, particularly in a vertical direction relative to the surface of the polymeric closure body 110. Moreover, the adhesion promotion layer material includes SiOxCy:H. In some embodiments, the at least one barrier layer 160 can have a sufficiently high degradation temperature to remain stable during subsequent processing, e.g., sterilization of the closure for pharmaceutical preparations 100, which can involve extreme temperatures.

In addition, the at least one barrier layer 160 comprises a barrier layer material within at least a portion of the thickness of the at least one barrier layer 160 distal from the surface of the polymeric closure body 110, particularly in a vertical direction relative to the surface of the polymeric closure body 110. Furthermore, the barrier layer material is selected from the group consisting of SixNy, SixCy, SixOyNz, SixOyCz, SiO₂, AlxOy, AlOxNy, TiO₂, and combinations thereof.

Due to the presence of an adhesion promotion layer material and a barrier layer material in the closure for pharmaceutical preparations 100 as described above, the closure for pharmaceutical preparations 100 of the present disclosure shows improved barrier properties, e.g., barrier properties against extractables or leachables, water vapor, and oxygen.

Further, due to the location of the adhesion promotion layer material and the barrier layer material within the at least one barrier layer 160 as described above, the at least one barrier layer 160 shows an appropriate balance between flexural rigidity and crack onset strain, i.e., fracture toughness, and thus the at least one barrier layer 160 can undergo significant levels of mechanical strain and match mechanical properties of the polymeric closure body 110. In particular, the adhesion promotion layer material provides an intermediate region between the barrier layer material and the polymeric closure body 110 that functions as a mechanical cushioning for mechanical strains.

In some embodiments, the polymeric closure body 110 may comprise at least one polymer selected from the group consisting of copolymer of styrene and butadiene, polyisoprene, halogenated copolymer of isobutylene and isoprene such as bromobutyl rubber or chlorobutyl rubber, and combinations thereof. In some embodiments, the polymeric closure body 110 can be an elastomeric closure body. In some embodiments, the polymeric closure body 110 may have a three-dimensional shape or structure. In some embodiments, the polymeric closure body 110 can be a stopper, a plunger, or a needle shield.

In some embodiments, the at least one barrier layer 160 may have a thickness equal to or smaller than 500 nm, particularly a thickness equal to or smaller than 480 nm, and more particularly a thickness equal to or smaller than 460 nm, and/or the at least one barrier layer 160 may have a thickness equal to or greater than 120 nm, particularly a thickness equal to or greater than 140 nm, and more particularly a thickness equal to or greater than 160 nm. In some embodiments, the at least one barrier layer 160 may have a thickness of 120 nm or above and of 500 nm or below, particularly a thickness of 140 nm or above and of 480 nm or below, and more particularly a thickness of 160 nm or above and of 460 nm or below.

In some embodiments, the at least one barrier layer 160 may have a water vapor transmission rate of less than 10⁻¹ g/m²/day, particularly less than 10⁻² g/m²/day, and more particularly 10⁻³ g/m²/day. Measurement of water vapor transmission rate may be performed according to ISO 2528:2017.

In some embodiments, the at least one barrier layer 160 may have an oxygen gas transmission rate of less than 10⁻¹ ml/m²/day, particularly less than 10⁻² ml/m²/day, and more particularly 10⁻³ ml/m²/day, at 23 °C and 0% relative humidity. Measurement of the oxygen gas transmission rate may be performed according to ISO 15105-1:2007.

In some embodiments, the at least one barrier layer 160 may include barrier properties against the migration of extractables, particularly leachables, from the polymeric closure body 110. In some embodiments, the extractables, particularly the leachables, may comprise at least one chemical of the group consisting of phthalates, trialkyl benzene-1, 2,4-tricarboxylates, and combinations thereof.

In some embodiments, the at least one barrier layer 160 may be directly disposed or deposited over the polymeric closure body 110. In some embodiments, no further layers or films are present between the polymeric closure body 110 and the at least one barrier layer 160.

In some embodiments, the barrier layer material may comprise SiO₂ and the at least one barrier layer 160 may comprise a first barrier layer over the polymeric closure body 110 with a gradient chemical composition across a thickness of the first barrier layer, particularly in a vertical direction relative to a surface of the polymeric closure body 110, as shown in an exemplary manner in FIG. 1.

Accordingly, as shown in an exemplary manner in FIG. 1, the at least one barrier layer 160 may correspond to a first barrier layer with a gradient chemical composition, wherein the gradient chemical composition is represented in an exemplary manner by a color transition across the thickness of the first barrier layer, particularly in a vertical direction related to a surface of the polymeric closure body. Accordingly, as shown in an exemplary manner in FIG. 1, a darkened section of the first barrier layer in FIG. 1 corresponds to a portion of the thickness of the first barrier layer proximal to the surface of the polymeric closure body 110 and a colorless section of the first barrier layer corresponds to a portion of the thickness of the first barrier layer distal from the surface of the polymeric closure body 110.

In some embodiments, the gradient chemical composition may comprise a first chemical composition corresponding to the adhesion promotion layer material within at least a portion of the thickness of the first barrier layer proximal to a surface of the polymeric closure body 110, particularly in a vertical direction relative to a surface of the polymeric closure body 110. In some embodiments, the gradient chemical composition may further comprise a second chemical composition corresponding to the barrier layer material within at least a portion of the thickness of the first barrier layer distal from the surface of the polymeric closure body 110, particularly in a vertical direction relative to a surface of the polymeric closure body 110.

In some embodiments, a surface of the polymeric closure body 110 onto which the at least one barrier layer is provided, e.g. deposited, may be treated to improve adhesion. Useful surface treatments may comprise electrical discharge in the presence of a suitable reactive or non-reactive atmosphere (e.g., plasma, glow discharge, corona discharge, dielectric barrier discharge or atmospheric pressure discharge), chemical pretreatment, or flame pretreatment.

FIG. 2 shows a schematic cross-sectional view of a portion of a closure for pharmaceutical preparations 200 according to further embodiments described herein. In FIG. 2, depiction of a whole closure for pharmaceutical preparations 200 similar to the closure for pharmaceutical preparations 100 in FIG. 1 has been omitted for the sake of simplicity.

According to some embodiments, which can be combined with other embodiments described herein, the closure for pharmaceutical preparations 200 includes a polymeric closure body 210. Further, the closure for pharmaceutical preparations 200 includes at least one barrier layer 260 over the polymeric closure body 210. Furthermore, the at least one barrier layer 260 comprises an adhesion promotion layer material within at least a portion of a thickness of the at least one barrier layer 260 proximal to a surface of the polymeric closure body 210, particularly in a vertical direction relative to the surface of the polymeric closure body 210.

In addition, the at least one barrier layer 260 comprises a barrier layer material within at least a portion of the thickness of the at least one barrier layer 260 distal from the surface of the polymeric closure body 210, particularly in a vertical direction relative to the surface of the polymeric closure body 210.

Further, as shown in an exemplary manner in FIG. 2, the at least one barrier layer 260 may comprise an adhesion promotion layer 215 over the polymeric closure body 210. Furthermore, the at least one barrier layer 260 may comprise a first barrier layer 220 over the adhesion promotion layer 215. In this particular example, the adhesion promotion layer 215 may comprise the adhesion promotion layer material.

According to some embodiments, which may be combined with other embodiments described herein, the first barrier layer 220 may comprise the barrier layer material.

In some embodiments, the adhesion promotion layer 215 may be directly disposed or deposited over the polymeric closure body 210. In some embodiments, the first barrier layer 220 may be directly disposed or deposited over the adhesion promotion layer 215. In some embodiments, no further layers or films are present between the polymeric closure body 210 and the adhesion promotion layer 215. In some embodiments, no further layers or films are present between the adhesion promotion layer 215 and the first barrier layer 220.

According to some embodiments, which may be combined with other embodiments described herein, the adhesion promotion layer 215 may have a thickness equal to or smaller than 350 nm, particularly a thickness equal to or smaller than 330 nm, and more particularly a thickness equal to or smaller than 310 nm. In some embodiments, the adhesion promotion layer 215 may have a thickness equal to or greater than 100 nm, particularly a thickness equal to or greater than 120 nm, and more particularly a thickness equal to or greater than 140 nm. In some embodiments, the adhesion promotion layer 215 may have a thickness of 100 nm or above and of 350 nm or below, particularly a thickness of 120 nm or above and of 330 nm or below, and more particularly a thickness of 140 nm or above and of 310 nm or below.

According to some embodiments, which may be combined with other embodiments described herein, the first barrier layer 220 may have a thickness equal to or smaller than 150 nm, particularly a thickness equal to or smaller than 130 nm, and more particularly a thickness equal to or smaller than 110 nm. In some embodiments, the first barrier layer 220 may have a thickness equal to or greater than 20 nm, particularly a thickness equal to or greater than 40 nm, and more particularly a thickness equal to or greater than 60 nm. In some embodiments, the first barrier layer 220 may have a thickness of 20 nm or above and of 150 nm or below, particularly a thickness of 40 nm or above and of 130 nm or below, and more particularly a thickness of 60 nm or above and of 110 nm or below.

FIGS. 3 to 5 show schematic cross-sectional views of a portion of a closure for pharmaceutical preparations 300, 400, 500 according to embodiments described herein. As also described above in relation to FIG. 2, depiction of a whole closure for pharmaceutical preparations 300, 400, 500 similar to the closure for pharmaceutical preparations 100 in FIG. 1 has been omitted for the sake of simplicity.

According to some embodiments, which can be combined with other embodiments described herein, the closure for pharmaceutical preparations 300, 400, 500 includes a polymeric closure body 310, 410, 510. Furthermore, the closure for pharmaceutical preparations 300, 400, 500 includes at least one barrier layer 360, 460, 560 over the polymeric closure body 310, 410, 510. Furthermore, the at least one barrier layer 360, 460, 560 comprises an adhesion promotion layer material within at least a portion of a thickness of the at least one barrier layer 360, 460, 560 proximal to a surface of the polymeric closure body 310, 410, 510, particularly in a vertical direction relative to the surface of the polymeric closure body 310, 410, 510.

In addition, the at least one barrier layer 360, 460, 560 comprises a barrier layer material within at least a portion of the thickness of the at least one barrier layer 360, 460, 560 distal from the surface of the polymeric closure body 310, 410, 510, particularly in a vertical direction relative to the surface of the polymeric closure body 310, 410, 510.

Furthermore, as shown in an exemplary manner in FIG. 3, the at least one barrier layer 360 may comprise an adhesion promotion layer 315 over the polymeric closure body 310. Furthermore, the at least one barrier layer 360 may comprise a first barrier layer 320 over the adhesion promotion layer 315. In addition, the at least one barrier layer 360 may comprise a second barrier layer 330 over the first barrier layer 320. In this particular example, the adhesion promotion layer 315 may comprise the adhesion promotion layer material.

According to some embodiments, which may be combined with other embodiments described herein, the second barrier layer 330 may comprise a barrier layer material. Further, the first barrier layer 320 and the second barrier layer 330 may comprise different barrier layer materials selected from the group consisting of SixNy, SixCy, SixOyNz, SixOyCz, SiO₂, AlxOy, AlOxNy, TiO₂, and combinations thereof. As an example, the first barrier layer 320 and the second barrier layer 330 may be in the following order: SixNy/SixCy.

In some embodiments, the second barrier layer 330 may be directly disposed or deposited over the first barrier layer 320. In some embodiments, no further layers or films are present between the first barrier layer 320 and the second barrier layer 330.

According to some embodiments, which may be combined with other embodiments described herein, the second barrier layer 330 may have a thickness equal to or smaller than 150 nm, particularly a thickness equal to or smaller than 130 nm, and more particularly a thickness equal to or smaller than 110 nm. In some embodiments, the second barrier layer 330 may have a thickness equal to or greater than 20 nm, particularly a thickness equal to or greater than 40 nm, and more particularly a thickness equal to or greater than 60 nm. In some embodiments, the second barrier layer 330 may have a thickness of 20 nm or above and of 150 nm or below, particularly a thickness of 40 nm or above and of 130 nm or below, and more particularly a thickness of 60 nm or above and of 110 nm or below.

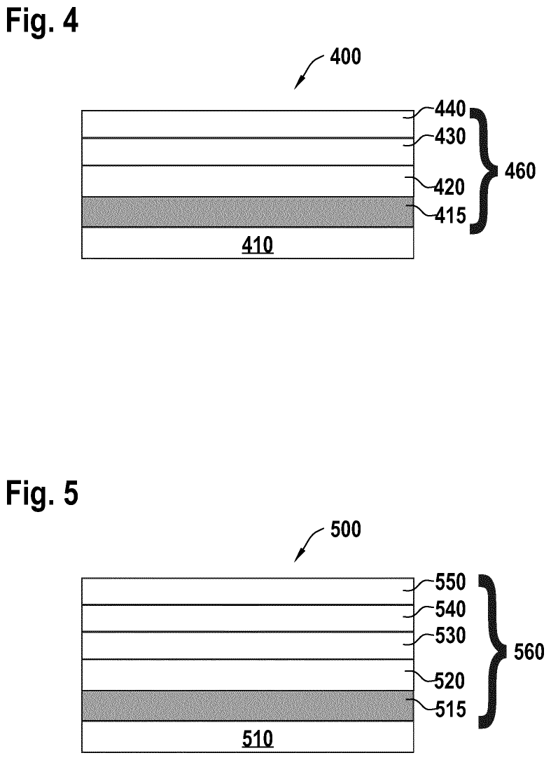

In FIG. 4, the closure for pharmaceutical preparations 400 is similar to the closure for pharmaceutical preparations 300 of FIG. 3, with the difference being that the at least one barrier layer 460 includes a further barrier layer, e.g., a third barrier layer 440.

Furthermore, as shown in an exemplary manner in FIG. 4, the at least one barrier layer 460 may comprise an adhesion promotion layer 415 over the polymeric closure body 410. Furthermore, the at least one barrier layer 460 may comprise a first barrier layer 420 over the adhesion promotion layer 415. In addition, the at least one barrier layer 460 may comprise a second barrier layer 430 over the first barrier layer 420. Moreover, the at least one barrier layer 460 may comprise a third barrier layer 440 over the second barrier layer 430. In this particular example, the adhesion promotion layer 415 may comprise the adhesion promotion layer material.

According to some embodiments, which may be combined with other embodiments described herein, the third barrier layer 440 may comprise a barrier layer material. Further, the first barrier layer 420, the second barrier layer 430, and the third barrier layer 440 may comprise different barrier layer materials selected from the group consisting of SixNy, SixCy, SixOyNz, SixOyCz, SiO₂, AlxOy, AlOxNy, TiO₂, and combinations thereof. As an example, the first barrier layer 420, the second barrier layer 430, and the third barrier layer 440 may be in the following order: SixNy/SixCy/SixOyNz.

In some embodiments, the second barrier layer 430 may be directly disposed or deposited over the first barrier layer 420. In some embodiments, the third barrier layer 440 may be directly disposed or deposited over the second barrier layer 430. In some embodiments, no further layers or films are present between the first barrier layer 420, the second barrier layer 430, and the third barrier layer 440.

According to some embodiments, which may be combined with other embodiments described herein, the third barrier layer 440 may have a thickness equal to or smaller than 150 nm, particularly a thickness equal to or smaller than 130 nm, and more particularly a thickness equal to or smaller than 110 nm. In some embodiments, the third barrier layer 440 may have a thickness equal to or greater than 20 nm, particularly a thickness equal to or greater than 40 nm, and more particularly a thickness equal to or greater than 60 nm. In some embodiments, the third barrier layer 440 may have a thickness of 20 nm or above and of 150 nm or below, particularly a thickness of 40 nm or above and of 130 nm or below, and more particularly a thickness of 60 nm or above and of 110 nm or below.

In FIG. 5, the closure for pharmaceutical preparations 500 is similar to the closure for pharmaceutical preparations 400 of FIG. 4, with the difference being that the at least one barrier layer 560 includes a further barrier layer, e.g., a fourth barrier layer 550.

Furthermore, as exemplary shown in FIG. 5, the at least one barrier layer 560 may comprise an adhesion promotion layer 515 over the polymeric closure body 510. Furthermore, the at least one barrier layer 560 may comprise a first barrier layer 520 over the adhesion promotion layer 515. In addition, the at least one barrier layer 560 may comprise a second barrier layer 530 over the first barrier layer 520. Moreover, the at least one barrier layer 560 may comprise a third barrier layer 540 over the second barrier layer 530. Furthermore, the at least one barrier layer 560 may comprise a fourth barrier layer 550 over the third barrier layer 540. In this particular example, the adhesion promotion layer 515 may comprise the adhesion promotion layer material.

According to some embodiments, which can be combined with other embodiments described herein, the fourth barrier layer 550 may comprise a barrier layer material. Further, the first barrier layer 520, the second barrier layer 530, the third barrier layer 540, and/or the fourth barrier layer 550 may comprise different barrier layer materials selected from the group consisting of SixNy, SixCy, SixOyNz, SixOyCz, SiO₂, AlxOy, AlOxNy, TiO₂, and combinations thereof. As an example, the first barrier layer 520, the second barrier layer 530, the third barrier layer 540, and the fourth barrier layer 550 may be in the following order: SixNy/SixCy/SixOyNz/SixOyCz.

In some embodiments, the second barrier layer 530 may be directly disposed or deposited over the first barrier layer 520. In some embodiments, the third barrier layer 540 may be directly disposed or deposited over the second barrier layer 530. In some embodiments, the fourth barrier layer 550 may be directly disposed or deposited over the third barrier layer 540. In some embodiments, no further layers or films are present between the first barrier layer 520, the second barrier layer 530, the third barrier layer 540, and the fourth barrier layer 550.

According to some embodiments, which may be combined with other embodiments described herein, the fourth barrier layer 550 may have a thickness equal to or smaller than 150 nm, particularly a thickness equal to or smaller than 130 nm, and more particularly a thickness equal to or smaller than 110 nm. In some embodiments, the fourth barrier layer 550 may have a thickness equal to or greater than 20 nm, particularly a thickness equal to or greater than 40 nm, and more particularly a thickness equal to or greater than 60 nm. In some embodiments, the fourth barrier layer 550 may have a thickness of 20 nm or above and of 150 nm or below, particularly a thickness of 40 nm or above and of 130 nm or below, and more particularly a thickness of 60 nm or above and of 110 nm or below.

According to some embodiments, which may be combined with other embodiments described herein, the at least one barrier layer 560 may comprise a second barrier layer 530 over the first barrier layer 520, may particularly comprise a second barrier layer 530 over the first barrier layer 520 and a third barrier layer 540 over the second barrier layer 530, and may more particularly comprise a second barrier layer 530 over the first barrier layer 520, a third barrier layer 540 over the second barrier layer 530, and a fourth barrier layer 550 over the third barrier layer 540.

According to some embodiments, which may be combined with other embodiments described herein, the at least one barrier layer 560 may include further barrier layers over the fourth barrier layer 550, particularly a fifth barrier layer, more particularly a fifth barrier layer and a sixth barrier layer. However, it is to be understood that the present disclosure is not limited to six barrier layers, and that any suitable number of barrier layers could be used for the at least one barrier layer 560.

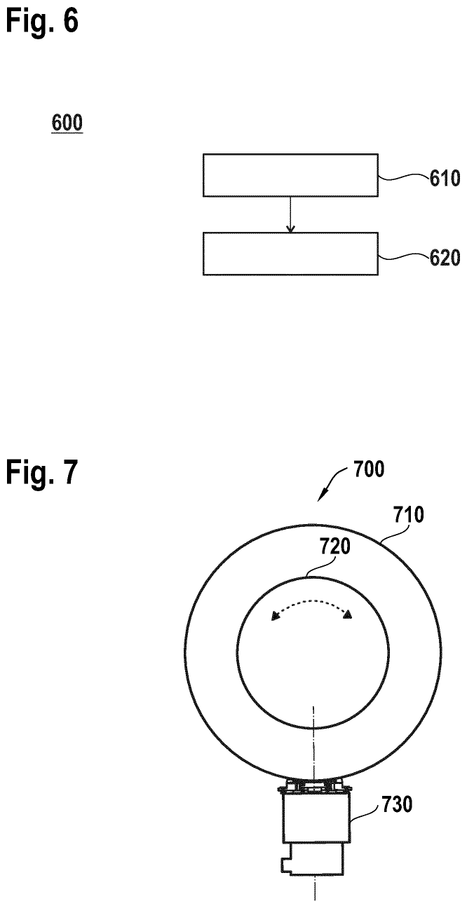

According to an aspect of the present disclosure and as shown in FIG. 6, a method 600 for manufacturing closures for pharmaceutical preparations is provided. The method 600 includes providing at least one polymeric closure body (stage 610). The method 600 further includes forming at least one barrier layer over the at least one polymeric closure body (stage 620).

Accordingly, forming at least one barrier layer over the at least one polymeric closure body includes depositing an adhesion promotion layer material and depositing at least one barrier layer material, in this order. Furthermore, the adhesion promotion layer material includes SiOxCy:H. Furthermore, the barrier layer material is selected from the group consisting of SixNy, SixCy, SixOyNz, SixOyCz, SiO₂, AlxOy, AlOxNy, TiO₂, and combinations thereof. Particularly, the term “in this order” together with the term “includes” in the sentence “forming at least one barrier layer over the at least one polymeric closure body includes depositing an adhesion promotion layer material and depositing at least one barrier layer material, in this order” may be understood as following a sequence of events in time, e.g., depositing an adhesion promotion layer material and thereafter depositing at least one barrier layer material, without excluding other sequences of the events in time, e.g., depositing an adhesion promotion layer material and depositing at least one barrier layer material simultaneously, e.g., between depositing an adhesion promotion layer material and thereafter depositing at least one barrier layer material.

Due to the simplicity of the deposition of the at least one barrier layer on the polymeric closure body, the method 600 for manufacturing closures for pharmaceutical preparations of the present disclosure allows a reduction of production costs and production time, and permits modulation of the barrier properties of the closure for pharmaceutical preparations by depositing further barrier layers with different barrier layer materials. Furthermore, the method 600 for manufacturing closures for pharmaceutical preparations of the present disclosure permits the formation of least one barrier layer over the at least one polymeric closure body even if the polymeric closure body has a three-dimensional shape or structure, particularly including portions of a surface of the polymeric closure body which are difficult to access.

In some embodiments, providing at least one polymeric closure body may comprise treating a surface of the at least one polymeric closure body, e.g., onto which the at least one barrier layer is formed or deposited, by applying an electrical discharge in the presence of a suitable reactive or non-reactive atmosphere (e.g., plasma, glow discharge, corona discharge, dielectric barrier discharge or atmospheric pressure discharge), a chemical pretreatment, or a flame pretreatment.

In some embodiments, forming at least one barrier layer over the at least one polymeric closure body, depositing an adhesion promotion layer material, and/or depositing at least one barrier layer material may be conducted by physical vapor deposition or chemical vapor deposition, for example sputtering or evaporation.

According to some embodiments, depositing an adhesion promotion layer material may comprise depositing an adhesion promotion layer material over the at least one polymeric closure body. According to some embodiments, depositing an adhesion promotion layer material may comprise forming an adhesion promotion layer, particularly over the at least one polymeric closure body. In some embodiments, depositing at least one barrier layer material may comprise depositing at least one barrier layer material over the adhesion promotion layer material. In some embodiments, depositing at least one barrier layer material may comprise depositing a first barrier layer material, particularly over the adhesion promotion layer material, and more particularly over the adhesion promotion layer. In some embodiments, depositing a first barrier layer material may comprise forming a first barrier layer.

In some embodiments, depositing at least one barrier layer may further comprise depositing a second barrier material, particularly over the first barrier layer material, and more particularly over the first barrier layer. In some embodiments, depositing a second barrier layer material may comprise forming a second barrier layer. In some embodiments, depositing at least one barrier layer may further comprise depositing a third barrier layer material, particularly over the second barrier layer material, and more particularly over the second barrier layer. In some embodiments, depositing a third barrier layer material may comprise forming a third barrier layer.

In some embodiments, depositing at least one barrier layer may further comprise depositing a fourth barrier layer material, particularly over the third barrier layer material, and more particularly over the third barrier layer. In some embodiments, depositing a fourth barrier layer material may comprise forming a fourth barrier layer. In some embodiments, the first barrier layer material, the second barrier layer material, the third barrier layer material, and/or the fourth barrier layer material may comprise different barrier layer materials selected from the group consisting of SixNy, SixCy, SixOyNz, SixOyCz, SiO₂, AlxOy, AlOxNy, TiO₂, and combinations thereof.

In some embodiments, depositing an adhesion promotion layer material may comprise introducing at least one adhesion promotion layer precursor into a plasma. In some embodiments, depositing a first barrier layer material may comprise introducing at least one first barrier layer precursor into a plasma. In some embodiments, depositing a second barrier layer material may comprise introducing at least one second barrier layer precursor into a plasma.

In some embodiments, depositing a third barrier layer material may comprise introducing at least one third barrier layer material precursor into a plasma. In some embodiments, depositing a fourth barrier layer material may comprise introducing at least one fourth barrier layer material precursor into a plasma. Examples of the first barrier layer material precursor, the second barrier layer material precursor, the third barrier layer material precursor, and the fourth barrier layer material precursor can be silanes, siloxanes, HMDSN (Hexamethyldisilazane), O₂, N₂O, NH₃, AlCl₃ (Aluminum trichloride), TMA (Trimethylaluminum), AlP (aluminum tri-isopropoxide), Titanium isopropoxide (Ti{OCH(CH₃)₂}₄), Titanium butoxide (C₁₆H₃₆O₄Ti), Titanium(IV) Acetylacetonate (Ti(C₅H₇O₂)₄). Examples of siloxanes are HMDSO (Hexamethyldisoxane), TMDSO (Tetamethyldisiloxane), and TEOS (Tetraethylorthosilicate).

In some embodiments, the first barrier layer material and the second barrier layer material may comprise different barrier layer materials selected from the group consisting of SixNy, SixCy, SixOyNz, SixOyCz, SiO₂, AlxOy, AlOxNy, TiO₂, and combinations thereof. In some embodiments, the first barrier layer material, the second barrier layer material, and the third barrier layer material may comprise different barrier layer materials selected from the group consisting of SixNy, SixCy, SixOyNz, SixOyCz, SiO₂, AlxOy, AlOxNy, TiO₂, and combinations thereof. In some embodiments, the first barrier layer material, the second barrier layer material, the third barrier layer material, and the fourth barrier layer material may comprise different barrier layer materials selected from the group consisting of SixNy, SixCy, SixOyNz, SixOyCz, SiO₂, AlxOy, AlOxNy, TiO₂, and combinations thereof.

According to some embodiments, the barrier layer material may comprise SiO₂ and forming the at least one barrier layer over the at least one polymeric closure body may further include decreasingly depositing the adhesion promotion layer material and increasingly depositing the at least one barrier layer material, particularly increasingly depositing a first barrier layer material, simultaneously.

In some embodiments, decreasingly depositing the adhesion promotion layer material and increasingly depositing the at least one barrier layer material, particularly increasingly depositing a first barrier layer material, may be conducted by gradually modifying at least one plasma-related process parameter while depositing the adhesion promotion layer material and/or gradually varying an amount or a chemical composition of at least one barrier layer material precursor, particularly of the first barrier layer material precursor, while depositing the adhesion promotion layer material.

Examples of the at least one barrier layer material precursors are silanes, siloxanes, HMDSN (Hexamethyldisilazane), O₂, N₂O, NH₃, AlCl₃ (Aluminum trichloride), TMA (Trimethylaluminum), AlP (aluminum tri-isopropoxide), Titanium isopropoxide (Ti{OCH(CH₃)₂}₄), Titanium butoxide (C₁₆H₃₆O₄Ti), Titanium(IV) Acetylacetonate (Ti(C₅H₇O₂)₄). Examples of siloxanes are HMDSO (Hexamethyldisoxane), TMDSO (Tetamethyldisiloxane), and TEOS (Tetraethylorthosilicate). Accordingly, decreasingly depositing the adhesion promotion layer material and increasingly depositing the at least one barrier layer material, particularly increasingly depositing a first barrier layer material may form a gradient chemical composition across a thickness of a first barrier layer.

In some embodiments, the at least one plasma-related process parameter may comprise at least one parameter selected from the group consisting of plasma power, carrier flow rate, temperature, bubbler flow rate, dilution flow rate, or plasma head vertical position relative to the polymeric closure body. Examples of apparatuses that may be used therefor are a classic bubbler or a mixing volatilize liquid delivery system.

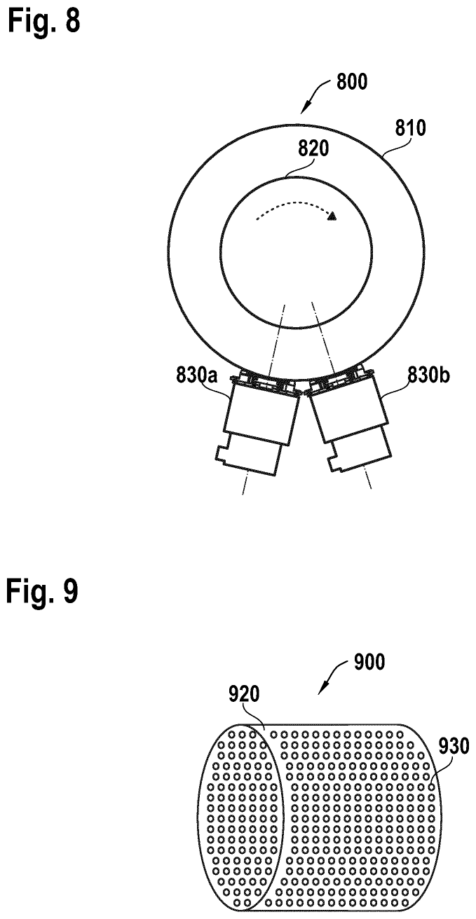

FIGS. 7 and 8 show schematic views of a rotary deposition apparatus 700 and 800 for manufacturing closures for pharmaceutical preparations according to embodiments described herein.

The rotary deposition apparatus 700 and 800 for manufacturing closures for pharmaceutical preparations includes a vacuum chamber 710, 810. Further, the rotary deposition apparatus 700 and 800 include a rotary container 720, 820 being located inside the vacuum chamber 710, 810 and configured to hold at least one polymeric closure body inside. Furthermore, the rotary deposition apparatus 700 and 800 include at least one deposition source 730,830a, 830b connected to the vacuum chamber 710, 810.

Moreover, the at least one deposition source 730, 830a, 830b connected to the vacuum chamber 710, 810 is located outside of the vacuum chamber 710, 810. Due to the location of the at least one deposition source 730, 830a, 830b, rapid cleaning, reparation, and replacement of parts of the at least one deposition source 730, 830a, 830b is possible, e.g., rapid cleaning of the shield and antenna-tube replacement of the at least one deposition source 730, 830a, 830b.

In addition, the at least one deposition source 730,830a, 830b connected to the vacuum chamber 710, 810 is configured for forming the at least one barrier layer on the at least one polymeric closure body, e.g., depositing an adhesion promotion layer material and/or depositing at least one barrier layer material. In addition, the rotary container 720, 820 includes a perforated wall configured to allow formation of the at least one barrier layer on the at least one polymeric closure body, e.g., depositing an adhesion promotion layer material and/or depositing at least one barrier layer material on the at least one polymeric closure body.

The rotary deposition apparatus for manufacturing closures for pharmaceutical preparations of the present disclosure allows manufacturing closures for pharmaceutical preparations on an industrial scale, i.e., in a high-volume manufacturing process, and at a high productivity level.

As shown in an exemplary manner in FIG. 7, the rotary deposition apparatus 700 may include one deposition source 730 connected to the vacuum chamber 710. Accordingly, the rotary deposition apparatus 700 including one deposition source 730 connected to the vacuum chamber 710 may form a gradient chemical composition across a thickness of a first barrier layer as described above, since it is possible to obtain a gradient chemical composition of this sort with only one deposition source.

As shown in an exemplary manner in FIG. 8, the rotary deposition apparatus 800 may include two deposition sources 830a, 830b connected to the vacuum chamber 810. Accordingly, the rotary deposition apparatus 800 including two deposition sources 830a, 830b connected to the vacuum chamber 810 may form at least one barrier layer including more than one barrier layer material as described above.

In some embodiments, the perforated wall can be centrosymmetric to a rotation axis of the rotary container 720, 820. In some embodiments, the rotary container 720, 820 may have a cylindrical shape, a spherical shape, or a cuboidal shape. In some embodiments, the rotary container 720, 820 may be configured to rotate clockwise and/or counterclockwise. Since the rotary deposition apparatus includes a rotary container, which causes a polymeric closure body or polymeric closure bodies to rotate inside the rotary container during manufacturing of the closures for pharmaceutical preparations, deposition of the at least one barrier layer over the polymeric closure body is homogenous over the whole polymeric closure body. Furthermore, deposition of the at least one barrier layer is conducted even on the portions of a surface of the polymeric closure body which are most difficult to reach, due to the combination of the rotary container and the at least one deposition source.

In some embodiments, the rotary container 720, 820 may be removable. Accordingly, the rotary container 720, 820 can be easily cleaned after a certain time period. In some embodiments, the at least one deposition source 730, 830a, 830b may include a high-density plasma chemical vapor deposition apparatus comprising an inductive coupled plasma apparatus or a microwave-induced plasma apparatus.

FIG. 9 shows a schematic view of a rotary container 900 of a rotary deposition apparatus for manufacturing closures for pharmaceutical preparations according to embodiments described herein.

In some embodiments, a perforated area of a perforated wall 920 with respect to a non-perforated area of the perforated wall 920 may be at least 75%, particularly at least 80%, more particularly at least 85%. In some embodiments, the perforated wall 920 may include circular, triangular, or square through-holes 930.

While the foregoing is directed to embodiments of the disclosure, other and further embodiments of the disclosure may be devised without departing from the basic scope thereof, and the scope thereof is determined by the claims that follow.

1. A closure for pharmaceutical preparations, comprising:
a polymeric closure body; and at least one barrier layer over the polymeric closure body,
wherein the at least one barrier layer comprises:
an adhesion promotion layer material within at least a portion of a thickness of the at least one barrier layer proximal to a surface of the polymeric closure body, the adhesion promotion layer material including SiOxCy:H, and a barrier layer material within at least a portion of the thickness of the at least one barrier layer distal from the surface of the polymeric closure body, the barrier layer material being selected from the group consisting of SixNy, SixCy, SixOyNz, SixOyCz, SiO₂, AlXOy, AlOxNy, TiO₂, and combinations thereof.

2. The closure for pharmaceutical preparations according to claim 1, wherein the barrier layer material comprises SiO₂, the at least one barrier layer includes a first barrier layer over the polymeric closure body with a gradient chemical composition across a thickness of the first barrier layer, and the gradient chemical composition includes:
a first chemical composition corresponding to the adhesion promotion layer material within at least a portion of the thickness of the first barrier layer proximal to a surface of the polymeric closure body, and
a second chemical composition corresponding to the barrier layer material within at least a portion of the thickness of the first barrier layer distal from the surface of the polymeric closure body.

3. The closure for pharmaceutical preparations according to claim 1, 5 wherein the at least one barrier layer comprises:
an adhesion promotion layer over the polymeric closure body; and a first barrier layer over the adhesion promotion layer, wherein the adhesion promotion layer comprises the adhesion promotion layer material and the first barrier layer comprises the barrier layer material.

4. The closure for pharmaceutical preparations according to claim 2, wherein the at least one barrier layer further comprises a second barrier layer over the first barrier layer.

5. The closure for pharmaceutical preparations according to claim 1, wherein the at least one barrier layer has at least one of a thickness equal to or smaller than 500 nm equal to or greater than 120 nm.

6. The closure for pharmaceutical preparations according to claim 1, wherein the at least one barrier layer has a water vapor transmission rate of less than 10⁻¹ g/m²/day, g/m/day, and more particularly 10 g/m/day.

7. The closure for pharmaceutical preparations according to claim 1, wherein the at least one barrier layer has an oxygen gas transmission rate of less than 10⁻¹ ml/m²/day, ml/m/day, and more particularly 10 ml/m/, at 23 °C and 0% relative humidity.

8. The closure for pharmaceutical preparations according to claim 1, wherein the polymeric closure body is an elastomeric closure body and the elastomeric closure body comprises at least one polymer selected from the group consisting of copolymer of styrene and butadiene, polyisoprene, halogenated copolymer of isobutylene and isoprene such as bromobutyl rubber or chlorobutyl rubber, and combinations thereof.

9. A method for manufacturing closures for pharmaceutical preparations comprising:
providing at least one polymeric closure body; and
forming at least one barrier layer over the at least one polymeric closure body, wherein forming at least one barrier layer over the at least one polymeric closure body includes depositing an adhesion promotion layer material and depositing at least one barrier layer material in this order, the adhesion promotion layer material includes SiOxCy:H, and the barrier layer material is selected from the group consisting of SixNy, SixCy, SixOyNz, SixOyCz, SiO₂, AlXOy, AlOxNy, TiO₂, and combinations thereof.

10. The method for manufacturing closures for pharmaceutical preparations according to claim 9, wherein the barrier layer material comprises SiO₂ and forming the at least one barrier layer over the at least one polymeric closure body further includes decreasingly depositing the adhesion promotion layer material and increasingly depositing the at least one barrier layer material simultaneously.

11. The method for manufacturing closures for pharmaceutical preparations according to claim 10, wherein decreasingly depositing the adhesion promotion layer material and increasingly depositing the at least one barrier layer material simultaneously comprises at least one of:
gradually modifying at least one plasma-related process parameter while depositing the adhesion promotion layer material, and gradually varying an amount or a chemical composition of at least one barrier layer material precursor while depositing the adhesion promotion layer material.

12. A rotary deposition apparatus for manufacturing closures for pharmaceutical preparations comprising:
a vacuum chamber;
a rotary container located inside the vacuum chamber and configured to hold at least one polymeric closure body inside; and at least one deposition source connected to the vacuum chamber, the at least one deposition source being located outside of the vacuum chamber and configured for forming at least one barrier layer on the at least one polymeric closure body, wherein the rotary container includes a perforated wall configured to allow forming the at least one barrier layer on the at least one polymeric closure body.

13. The rotary deposition apparatus according to claim 12, wherein a perforated area of the perforated wall with respect to a non-perforated area of the perforated wall is at least 75%.

14. The rotary deposition apparatus according to claim 12, and wherein the perforated wall is centrosymmetric to a rotation axis of the rotary container.

15. The rotary deposition apparatus according to claim 12, wherein the at least one deposition source includes a high-density plasma chemical vapor deposition apparatus comprising an inductive coupled plasma apparatus or a microwave-induced plasma apparatus.

16. The closure for pharmaceutical preparations according to claim 3, wherein the at least one barrier layer further comprises a second barrier layer over the first barrier layer.

17. The closure for pharmaceutical preparations according to claim 4, wherein the at least one barrier layer further comprises a second barrier layer over the first barrier layer and a third barrier layer over the second barrier layer.

18. The closure for pharmaceutical preparations according to claim 4, wherein the at least one barrier layer further comprises a second barrier layer over the first barrier layer, a third barrier layer over the second barrier layer, and a fourth barrier layer over the third barrier layer.
